(19) United States
(12) Patent Application Publication
(10) Pub. No.: US 2025/0269192 A1
(43) Pub. Date: Aug. 28, 2025

(54) CONTROL METHOD FOR AN INTRACARDIAC DEFIBRILLATION CATHETER SYSTEM

(71) Applicant: KANEKA CORPORATION, Osaka (JP)

(72) Inventor: Toshiya Kisa, Osaka (JP)

(73) Assignee: KANEKA CORPORATION, Osaka (JP)

(21) Appl. No.: 19/205,237

(22) Filed: May 12, 2025

Related U.S. Application Data
(63) Continuation of application No. PCT/JP2023/040186, filed on Nov. 8, 2023.

(30) Foreign Application Priority Data
Nov. 15, 2022 (JP) 2022-182528

(51) Int. Cl. A61N 1/39

(52) U.S. Cl. CPC A61N 1/3906; A61N 1/3968; A61N 1/3975

(57) ABSTRACT

A control method for an intracardiac defibrillation catheter system is provided for performing a safer treatment by shortening the time between switching the mode to perform defibrillation and synchronization with R wave. The system includes a catheter, electrode part, power supply, input part inputting an electrocardiogram, and changeover part connected to the power supply, for switching between a first mode and second mode for electrically disconnecting and connecting the power supply and electrode part, which is connected to the power supply via the changeover part. The method includes switching the first mode to second mode with the changeover part when a cardiac potential reaches a peak of P wave and/or Q wave obtained from an electrocardiographic waveform of the electrocardiogram, and applying a voltage to the electrode part with the power supply when the cardiac potential reaches a peak of R wave obtained from the electrocardiographic waveform of the electrocardiogram.

### TECHNICAL FIELD

One or more embodiments of the present invention relate to a control method for an intracardiac defibrillation catheter system used for defibrillation in the heart cavity.

### BACKGROUND

In the treatment of arrhythmia such as atrial fibrillation or ventricular fibrillation, defibrillation is performed to restore the heart rhythm to normal by applying electrical stimulation. For the defibrillation, an automated external defibrillator (AED), an implantable cardioverter defibrillator (ICD), a defibrillation paddle, an intracardiac defibrillation catheter system, and the like are used. The intracardiac defibrillation catheter system is a device that directly applies electrical stimulation to the heart through an electrode provided on the surface of a catheter. According to the intracardiac defibrillation catheter system, the intracardiac potential can also be measured by the electrode. The defibrillation catheter system is advantageous in that it can use a voltage waveform with lower energy than an automated external defibrillator, by which the burden on a patient is reduced. The defibrillation catheter system is also advantageous in that it can also be used during ablation and cardiac catheterization for diagnosis of arrhythmia.

In the treatment of atrial fibrillation, it is necessary to apply a voltage to the heart during an absolute refractory period so that the ventricular muscles do not respond. In a case where stimulation is applied to the heart during periods other than the absolute refractory period, ventricular muscles may respond to cause ventricular fibrillation. Therefore, the defibrillation catheter system applies a voltage in synchronization with an R wave. Patent Document 1 describes, as an example of such a defibrillation catheter system, a system including: a catheter extending in a distal and proximal direction; a power supply part that is connected to the catheter and that generates a voltage to be applied; and an electrocardiograph that measures an intracardiac potential, wherein a first electrode and a second electrode are provided on a distal side of the catheter, the second electrode being located proximal to the first electrode, a changeover part is connected to the power supply part, the changeover part switching between a first mode for measuring the intracardiac potential and a second mode for applying the voltage while measuring the intracardiac potential, the first electrode and the second electrode are connected to the power supply part via the changeover part, and the first electrode and the second electrode are connected to the electrocardiograph without a switching part.

### PATENT DOCUMENT

Patent Document 1: WO2019/155941A

In a case where the intracardiac defibrillation catheter system is used for defibrillation, the intracardiac defibrillation catheter system applies a voltage to the heart in synchronization with the R wave after switching from a mode for measuring the cardiac potential to a mode for performing defibrillation. In this case, the cardiac potential is not measured from the time point at which the mode for measuring the cardiac potential is switched to the mode for performing defibrillation until the synchronization with the R wave is achieved. Therefore, the timing of defibrillation is shifted, resulting in a voltage not being applied to the heart in an absolute refractory period.

### SUMMARY

One or more embodiments of the present invention have been made in view of such circumstances to provide a control method for an intracardiac defibrillation catheter system with which it is possible to perform a safer treatment by shortening the time from switching to a mode for performing defibrillation to synchronization with the R wave.

One or more embodiments of the present invention are configured as follows.

[1]A control method for an intracardiac defibrillation catheter system comprising:
a catheter extending from a proximal side to a distal side in a longitudinal direction;
an electrode part disposed on a distal side of the catheter;
a power supply for applying a voltage to the electrode part;
an input part for inputting an electrocardiogram; and
a changeover part for switching between a first mode for electrically disconnecting the power supply and the electrode part, and a second mode for electrically connecting the power supply and the electrode part, the changeover part being connected to the power supply, and the electrode part being connected to the power supply via the changeover part,
the control method comprising:
switching the first mode to the second mode with the changeover part when a cardiac potential reaches a peak of a P wave and/or a peak of a Q wave obtained from an electrocardiographic waveform of the electrocardiogram; and
applying a voltage to the electrode part with the power supply when the cardiac potential reaches a peak of an R wave obtained from the electrocardiographic waveform of the electrocardiogram.

[2] The control method according to [1], wherein the intracardiac defibrillation catheter system further comprises an electrocardiograph connected to the input part.

[3] The control method according to [2], wherein the electrocardiograph comprises an electrocardiogram detection unit detecting a peak of a P wave and/or a peak of a Q wave and a peak of an R wave from the electrocardiographic waveform of the electrocardiogram obtained from the cardiac potential.

[4] The control method according to [2] or [3], wherein the electrocardiograph is connected to the electrode part.

[5] The control method according to any one of [1] to [4], wherein the electrode part includes an electrode A and an electrode B located proximal to the electrode A.

[6] The control method according to [5], wherein
the changeover part includes a switch A and a switch B,
the electrode A is connected to the power supply via the switch A, and
the electrode B is connected to the power supply via the switch B.

[7] The control method according to [5] or [6], wherein
the electrode A includes a plurality of electrodes a,
the electrode B includes a plurality of electrodes b,
the changeover part includes a plurality of switches a connected in parallel to each other and a plurality of switches b connected in parallel to each other,
each of the plurality of electrodes a is connected to the power supply via a corresponding one of the plurality of switches a, and
each of the plurality of electrodes b is connected to the power supply via a corresponding one of the plurality of switches b.

[8] The control method according to [7], wherein the intracardiac defibrillation catheter system further comprises an electrode selection switch connected to the power supply for selecting an electrode to which the voltage is applied.

[9] The control method according to any one of [2] to [8], wherein the intracardiac defibrillation catheter system further comprises a switch disposed in a connection path between the electrode part and the electrocardiograph.

[10] The control method according to any one of [2] to [9], wherein the intracardiac defibrillation catheter system further comprises a resistor of 200Ω or less disposed in a connection path between the electrode part and the electrocardiograph.

[11] The control method according to any one of [2] to [10], wherein the electrocardiograph is connected to the power supply, and the intracardiac defibrillation catheter system further comprises an overvoltage protection circuit provided in a connection path between the power supply and the electrocardiograph for protecting the electrocardiograph from overvoltage.

[12] The control method according to any one of [1] to [11], wherein the intracardiac defibrillation catheter system further comprises a power supply output control part disposed in a connection path between the power supply and the changeover part.

According to one or more embodiments of the present invention, when the cardiac potential reaches the peak of the P wave and/or the peak of the Q wave obtained from the electrocardiographic waveform of the electrocardiogram, the changeover part connected to the power supply switches the first mode for electrically disconnecting the power supply and the electrode part to the second mode for electrically connecting the power supply and the electrode part. Therefore, when the cardiac potential reaches the peak of the R wave obtained from the electrocardiographic waveform of the electrocardiogram, the power supply can quickly apply a voltage to the electrode part. As a result, the time from switching to the second mode (the mode for performing defibrillation) to the execution of defibrillation can be shortened, whereby safer treatment can be performed.

### BRIEF DESCRIPTION OF THE DRAWINGS

FIG. 1 is a schematic diagram showing an intracardiac defibrillation catheter system according to one or more embodiments.

FIG. 2 shows an example of an electrocardiogram obtained from the intracardiac potential measured by an electrocardiograph.

FIG. 3 shows an example of a circuit diagram of an intracardiac defibrillation catheter system.

FIG. 4 shows another example of a circuit diagram of an intracardiac defibrillation catheter system.

FIG. 5 shows another example of a circuit diagram of an intracardiac defibrillation catheter system.

### DETAILED DESCRIPTION

One or more embodiments of the present invention will be described below in more detail by way of the following embodiments. However, one or more embodiments of the present invention are not limited to the following embodiments. It is obvious that one or more embodiments of the present invention can be carried out by making modifications in accordance with the gist described above or later, and such modifications are also included in the technical scope of one or more embodiments of the present invention. Here, the proximal side indicates a side closer to a user's (operator's) hand, and the distal side indicates the side opposite to the proximal side (i.e., the side closer to a treatment target). In each drawing, hatching, reference signs for components, and the like may be omitted for convenience of description, and in such a case, the specification and other drawings are to be referred to. The dimensions of the various components in the drawings are provided for the purpose of facilitating the understanding of the feature of one or more embodiments of the present invention, and the dimensions may differ from the actual dimensions in some cases.

The intracardiac defibrillation catheter system to be controlled with the control method for an intracardiac defibrillation catheter system according to one or more embodiments of the present invention includes a catheter extending from a proximal side to a distal side in a longitudinal direction, an electrode part disposed on a distal side of the catheter, a power supply applying a voltage to the electrode part, and an input part inputting an electrocardiogram. The intracardiac defibrillation catheter system also includes a changeover part for switching between a first mode for electrically disconnecting the power supply and the electrode part and a second mode for electrically connecting the power supply and the electrode part, the changeover part being connected to the power supply. The electrode part is connected to the power supply via the changeover part. The control method for an intracardiac defibrillation catheter system according to one or more embodiments of the present invention includes: switching the first mode to the second mode with the changeover part when a cardiac potential reaches a peak of a P wave and/or a peak of a Q wave obtained from an electrocardiographic waveform of the electrocardiogram; and applying a voltage to the electrode part with the power supply when the cardiac potential reaches a peak of an R wave obtained from the electrocardiographic waveform of the electrocardiogram.

The control method for an intracardiac defibrillation catheter system according to one or more embodiments of the present invention includes switching the first mode for electrically disconnecting the power supply and the electrode part to the second mode (defibrillation mode) for electrically connecting the power supply and the electrode part by the changeover part when a cardiac potential reaches the peak of the P wave and/or the peak of the Q wave obtained from an electrocardiographic waveform of the electrocardiogram, prior to the application of voltage to the electrode part with the power supply to execute defibrillation. Therefore, when the defibrillation is performed, the power supply can apply a voltage to the electrode part at the timing at which the cardiac potential reaches the peak of the R wave to perform the defibrillation. As a result, the time to the execution of defibrillation in synchronization with the R wave can be shortened, whereby safer treatment can be performed. One or more embodiments of the present invention will be described below in detail.

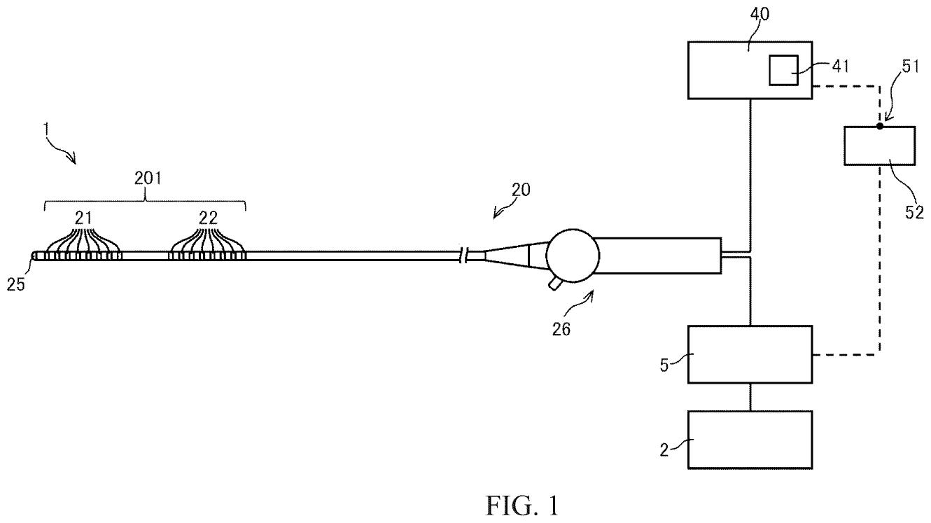

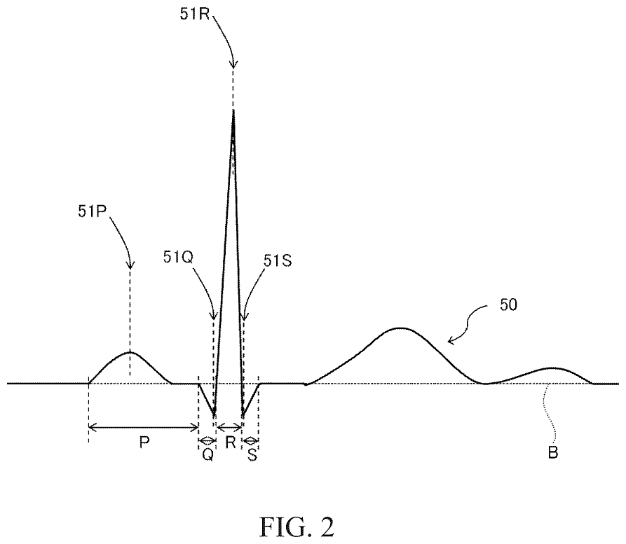

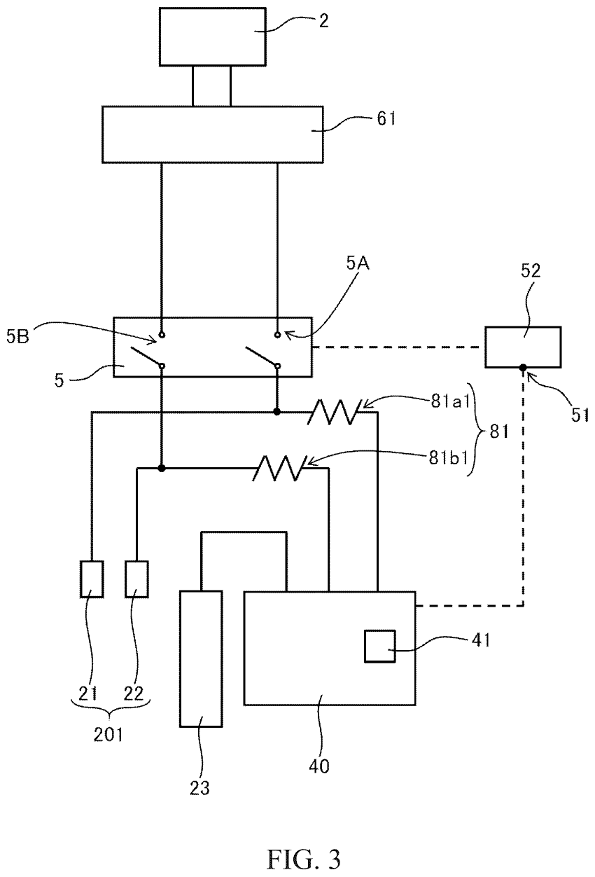

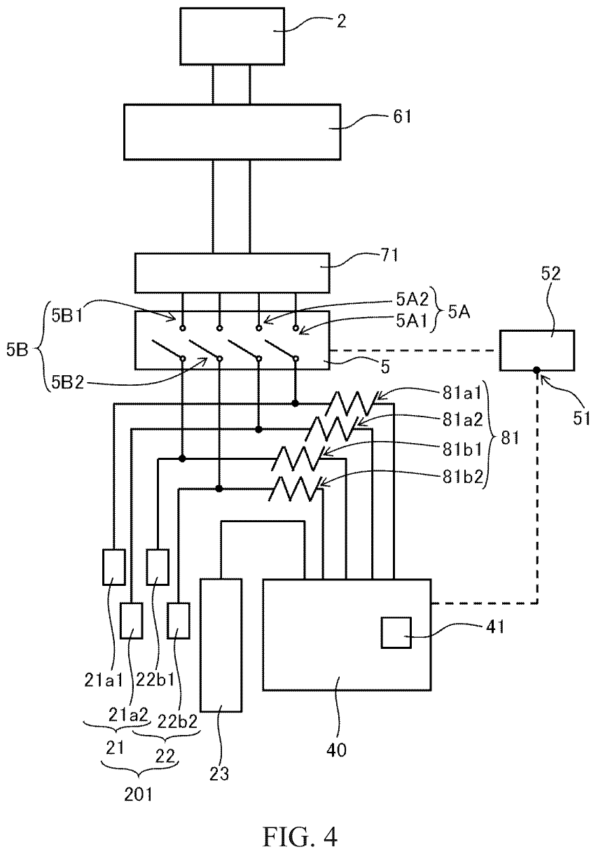

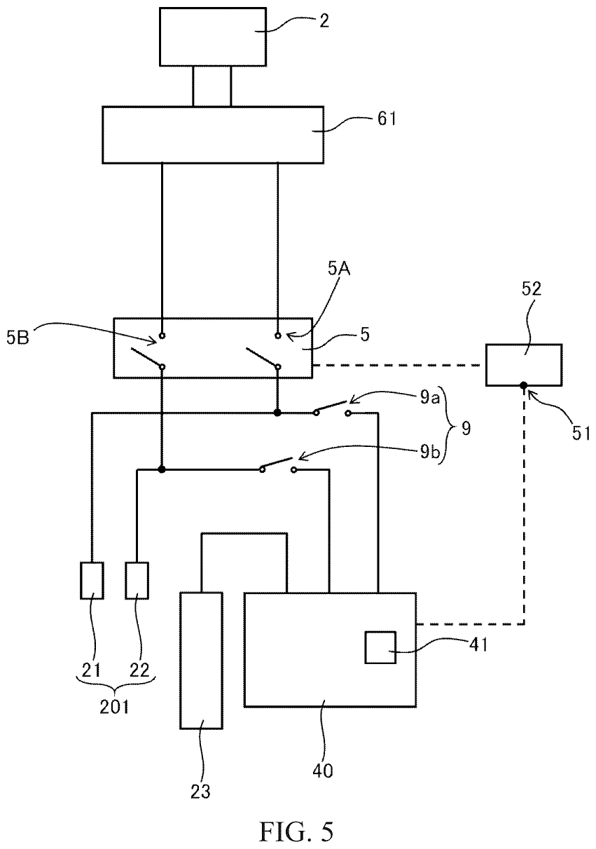

The control method of the intracardiac defibrillation catheter system according to one or more embodiments of the present invention will be described with reference to FIGS. 1 to 5. FIG. 1 is a schematic diagram showing an embodiment of an intracardiac defibrillation catheter system that is controlled by the control method for the intracardiac defibrillation catheter system according to one or more embodiments of the present invention. FIG. 2 shows an example of an electrocardiogram obtained from the intracardiac potential measured by an electrocardiograph. In FIG. 2, the horizontal axis shows time (in seconds), and the vertical axis shows voltage difference (in mV). FIG. 3 shows an example of a circuit diagram of an intracardiac defibrillation catheter system that is controlled by the control method for the intracardiac defibrillation catheter system according to one or more embodiments of the present invention. FIG. 4 shows another example of a circuit diagram of an intracardiac defibrillation catheter system that is controlled by the control method for the intracardiac defibrillation catheter system according to one or more embodiments of the present invention. FIG. 5 shows another example of a circuit diagram of an intracardiac defibrillation catheter system that is controlled by the control method for the intracardiac defibrillation catheter system according to one or more embodiments of the present invention.

An intracardiac defibrillation catheter system 1 illustrated in FIG. 1 includes a catheter 20 extending from the proximal side to the distal side in the longitudinal direction. The longitudinal direction refers to a direction from the proximal side to the distal side of the catheter 20. The proximal side of the catheter 20 indicates a side closer to a user's (operator's) hand with respect to the extending direction of the catheter 20, and the distal side indicates the side opposite to the proximal side (i.e., the side closer to a treatment target). An electrode part 201 is disposed on the distal side of the catheter 20. The electrode part 201 includes at least one pair of a positive electrode and a negative electrode.

Preferably, the intracardiac defibrillation catheter system to be controlled with the control method for an intracardiac defibrillation catheter system according to one or more embodiments of the present invention has an input part 51 that inputs an electrocardiogram, and the input part 51 is disposed in a control part 52. The control part 52 functions to control a changeover part 5 such that the changeover part 5 switches from a first mode to a second mode based on the electrocardiogram input from the input part 51. The input part 51 may be a part of a circuit constituting the control part 52, or may be an input terminal physically connected to a device that measures an electrocardiogram based on a cardiac potential. The input part 51 may be connected to, for example, an electrocardiograph 40. The electrocardiogram input from the input part 51 may be obtained based on an intracardiac potential measured from an electrode inserted into the heart, or may be obtained based on a body surface potential measured from an electrode attached to the body surface. The control part 52 may be connected to a power supply 2. Due to the control part 52 being connected to the power supply 2, the power supply 2 can be controlled based on the electrocardiogram input from the input part 51.

As illustrated in FIGS. 1 and 3 to 5, the electrocardiograph 40 may be connected to the electrode part 201. The electrocardiograph 40 is a device that generates an electrocardiographic waveform based on the intracardiac potential measured by the electrode part 201. Since the electrocardiograph 40 is provided, the electrocardiographic waveform can be obtained based on the intracardiac potential measured with the electrode part 201 by inserting the catheter 20 into the cardiac cavity and bringing the electrode part 201 into contact with the inner surface of the atrium, the ventricle, or the blood vessel. The electrocardiograph 40 may include an electrocardiogram detection unit 41 detecting a peak of a P wave and/or a peak of a Q wave and a peak of an R wave from the electrocardiographic waveform obtained from the intracardiac potential measured by the electrocardiograph 40. A known device can be used as the electrocardiograph 40.

The power supply 2 is connected to the electrode part 201 and applies a voltage to the electrode part 201, so that the heart can be stimulated, and defibrillation can be performed. The changeover part 5 is connected to the power supply 2, and the power supply 2 is connected to the electrode part 201 via the changeover part 5. The changeover part 5 can switch between the first mode for electrically disconnecting the power supply 2 and the electrode part 201 and the second mode for electrically connecting the power supply 2 and the electrode part 201.

The control method for an intracardiac defibrillation catheter system according to one or more embodiments of the present invention includes: switching the first mode for electrically disconnecting the power supply 2 and the electrode part 201 to the second mode for electrically connecting the power supply 2 and the electrode part 201 by the changeover part 5 when a cardiac potential reaches the peak of the P wave and/or the peak of the Q wave obtained from an electrocardiographic waveform of the electrocardiogram; and applying a voltage to the electrode part 201 by the power supply 2 when the cardiac potential reaches the peak of the R wave obtained from the electrocardiographic waveform of the electrocardiogram. Whether or not the cardiac potential reaches the peak of the P wave, the peak of the Q wave, or the peak of the R wave obtained from the electrocardiographic waveform of the electrocardiogram may be determined by analyzing the electrocardiogram input from the input part 51 or may be detected by analyzing the electrocardiographic waveform obtained from the intracardiac potential measured by the electrocardiograph 40 provided with the electrocardiogram detection unit 41 as illustrated in FIGS. 1 and 3 to 5.

FIG. 2 illustrates an example of an electrocardiographic waveform obtained from an intracardiac potential measured by the electrocardiograph 40. In an electrocardiographic waveform 50 illustrated in FIG. 2, a wave that appears first and has an upward deflection with respect to a baseline B is a P wave. The P wave indicates atrial activation. A wave that appears next to the P wave and has a downward deflection with respect to the baseline B is a Q wave.

A wave that appears next to the Q wave and has an upward deflection with respect to the baseline B is an R wave. The peak height of the R wave is the maximum as compared with other waves. A wave that appears next to the R wave and has a downward deflection with respect to the baseline B is an S wave. The period from the beginning of the Q wave to the end of the S wave is called a QRS wave, and indicates activation of both left and right ventricular muscles. In the following, the peak position of the P wave is defined as 51P, the peak position of the Q wave is defined as 51Q, the peak position of the R wave is defined as 51R, and the peak position of the S wave is defined as 51S. Each of the P wave, the Q wave, the R wave, and the S wave may be distinguished by setting a threshold for signal intensity (height), width, slope (differential waveform), and the like of the electrocardiographic waveform, and a peak position of each wave may be determined. Alternatively, focusing on the R wave, the waves may be distinguished in a section estimated from an interval at which the R wave is detected, and a peak position of each wave may be determined.

In the control method according to one or more embodiments of the present invention, when the electrocardiogram detection unit 41 detects the peak 51P of the P wave and/or the peak 51Q of the Q wave, the changeover part 5 switches from the first mode for electrically disconnecting the power supply 2 and the electrode part 201 to the second mode for electrically connecting the power supply 2 and the electrode part 201. As a result, when the peak 51R of the R wave is detected by the electrocardiogram detection unit 41, the power supply 2 can quickly apply a voltage to the electrode part 201, so that it is possible to shorten the time from when the mode is switched to the second mode for enabling defibrillation until the defibrillation is performed by applying the voltage to the electrode part 201. Thus, a safer treatment can be performed.

The changeover part 5 switches from the first mode to the second mode at a timing at which the peak 51P of the P wave is detected or at a timing at which the peak 51Q of the Q wave is detected, or at a timing at which both the P wave and the Q wave are detected. Since there is a possibility of erroneous detection at one of the peak 51P of the P wave and the peak 51Q of the Q wave, the changeover part 5 may switch from the first mode to the second mode at a timing at which both the peak 51P of the P wave and the peak 51Q of the Q wave are detected. Thus, a safer treatment can be performed.

The power supply 2 applies a voltage to the electrode part 201 at a timing at which the peak 51R of the R wave is detected.

Preferably, deep learning by Artificial Intelligence (AI), for example, is used to detect the peak 51P of the P wave, the peak 51Q of the Q wave, and the peak 51R of the R wave. For example, a plurality of electrocardiographic waveforms acquired for a certain period of time may be learned, characteristics of the patient such as P waves and Q waves may be estimated, and the P waves, the Q waves, and the like may be detected using the estimated parameters.

One or more embodiments of the intracardiac defibrillation catheter system to be controlled with the control method for an intracardiac defibrillation catheter system according to one or more embodiments of the present invention will be described below more specifically.

As the catheter 20, a resin tube formed in a tubular shape can be used. Examples of resin constituting the resin tube include polyamide resin, polyester resin, polyurethane resin, polyolefin resin, fluorine resin, vinyl chloride resin, silicone resin, and natural rubber.

These materials may be used alone, or two or more of them may be used in combination. Among them, polyamide resin, polyester resin, polyurethane resin, polyolefin resin, and fluorine resin may be used. The resin tube can be manufactured by, for example, extrusion molding.

The catheter 20 may have a single-layer structure or a multi-layer structure. In addition, the catheter 20 may have a portion having a single-layer structure and a portion having a multi-layer structure. For example, the catheter 20 may be configured such that a part in the longitudinal direction or in the circumferential direction is composed of a single layer, and another part is composed of a plurality of layers.

The catheter 20 may have a lumen. The number of lumens is not particularly limited, and may be one or two or more.

An electrode part 201 is disposed on the distal side of the catheter 20. The electrode part 201 includes at least a pair of electrodes. When one of the electrodes is defined as an electrode A and the other is defined as an electrode B, the electrode part 201 may include the electrode A and the electrode B located proximal to the electrode A in the longitudinal direction of the catheter 20.

The electrode A and the electrode B are arranged to be shifted in the longitudinal direction of the catheter 20. Thus, even when not the entire circumference but only a part of the catheter 20 contacts the inner surface of the atrium, the ventricle, or the blood vessel, at least a part of the electrode A and at least a part of the electrode B are likely to be in contact with the inner surface of the atrium, the ventricle, or the blood vessel, whereby the intracardiac potential can be easily measured. In addition, when at least a part of the electrode A and at least a part of the electrode B are in contact with the inner surface of the atrium, ventricle, or blood vessel, the heart can be stimulated by application of a voltage from the power supply 2 to the electrode A and the electrode B, and defibrillation can be performed. Specifically, a voltage is applied such that a current flows from the electrode A to the electrode B via a living body or from the electrode B to the electrode A via the living body.

It is preferable that, when the catheter 20 including the electrode A and the electrode B is inserted into the cardiac cavity, the electrode A is placed in a position corresponding to the coronary sinus, and the electrode B is placed in a position corresponding to the right atrium. Due to the electrode A and the electrode B being placed as described above, atrial fibrillation can be terminated efficiently.

DC voltages of different polarities may be applied to the electrode A and the electrode B. For example, when a biphasic DC voltage is applied, defibrillation can be performed with a small amount of energy.

The surface area of the electrode A and the surface area of the electrode B may be different from each other, or may be the same. By setting the electrode A and the electrode B to have the same surface area, the accuracy in measurement of the intracardiac potential can be enhanced.

The width of the electrode A and the width of the electrode B in the longitudinal direction of the catheter 20 may be different from each other, or may be the same. By setting the electrode A and the electrode B to have the same width, the accuracy in measurement of the intracardiac potential can be enhanced. The width of each electrode may be, for example, 0.5 mm or more and 5 mm or less.

The electrode A may be a positive electrode or a negative electrode. When the electrode A is a positive electrode, the electrode B may be a negative electrode, and when the electrode A is a negative electrode, the electrode B may be a positive electrode. Preferably, the electrode A is a positive electrode, and the electrode B is a negative electrode.

The number of each of the electrodes A and B may be one, but preferably, the electrode A is constituted by a plurality of electrodes a and the electrode B is constituted by a plurality of electrodes b. By arranging the plurality of electrodes a and the plurality of electrodes b, the intracardiac potential can be measured at various positions. For example, the intracardiac potential between the adjacent electrodes a can be measured by measuring the potential difference between the electrodes a. The same applies to the electrodes b. Furthermore, since the plurality of electrodes a and the plurality of electrodes b are arranged, a voltage can be applied to a wide area of the heart, so that defibrillation can be efficiently performed.

Voltages of the same polarity (positive or negative) may be applied to the plurality of electrodes a, and voltages of the same polarity (negative or positive) may be applied to the plurality of electrodes b. For example, in the case of applying a biphasic DC voltage, the electrodes a are negative and the electrodes b are positive in the first half of current application, so that a current flows from the right atrium toward the coronary sinus, and in the latter half of the current application, the electrodes a are positive and the electrodes b are negative, so that a current flows from the coronary sinus toward the right atrium.

In a case where the plurality of electrodes a is arranged, the electrode b may be placed proximal to the most proximal electrode a. Due to the electrodes a and b being placed as described above, defibrillation can be efficiently performed.

When the plurality of electrodes a and the plurality of electrodes b are arranged, the number of electrodes a and the number of electrodes b are not particularly limited, and may be different or the same. By setting the number of electrodes a to be the same as the number of electrodes b, it is easy to set the total surface area of the plurality of electrodes a to be the same as the total surface area of the plurality of electrodes b.

The same number of electrodes are equally arranged and the total surface area of the plurality of electrodes a is set to be the same as the total surface area of the plurality of electrodes b, whereby it is possible to improve the accuracy in measurement of the intracardiac potential and to efficiently perform defibrillation.

In a case where the plurality of electrodes a and the plurality of electrodes b are arranged, the number of the electrodes a and the number of the electrodes b may be each 6 or more, for example, or 8 or more, and 12 or less, or 10 or less. In a case where the plurality of electrodes a and the plurality of electrodes b are arranged, the width of each of the electrodes in the longitudinal direction of the catheter 20 may be different, or may be the same. The width of each electrode may be, for example, 0.5 mm or more and 5 mm or less. In a case where the plurality of electrodes a and the plurality of electrodes b are arranged, the spacing between adjacent electrodes, that is, the distance between the distal end of one electrode and the proximal end of the other electrode that is located distal to one electrode can be set to, for example, 1 mm or more and 10 mm or less, or set to 3 mm or more and 8 mm or less. By setting the distance between the electrodes and the electrode width as described above, the accuracy in measurement of intracardiac potential can be improved. In a case where the plurality of electrodes a and the plurality of electrodes b are arranged, the electrodes have the same width in the longitudinal direction of the catheter 20, and the total surface area of the plurality of electrodes a and the total surface area of the plurality of electrodes b are the same, whereby the contact conditions with the myocardium can be made the same. The electrode width indicates the length of each electrode in the longitudinal direction.

Each electrode may be present in at least a half region of the outer circumference of the resin tube, or each electrode may be formed in a ring shape in a region covering the entire outer circumference of the resin tube. When each electrode is formed in the manner described above, the contact area between each electrode and the heart increases, which facilitates the measurement of intracardiac potential and application of electrical stimulation.

Each electrode may contain a conductive material such as platinum or stainless steel, or may contain a radio-opaque material among the conductive materials. The radio-opaque material may contain, for example, platinum. When each electrode contains the radio-opaque material, the position of the electrode can be easily recognized under radioscopy.

In a case where the electrode A is constituted by a plurality of electrodes a and the electrode B is constituted by a plurality of electrodes b, it is preferable to dispose a wiring connecting part 71 between the changeover part 5 and the power supply 2 as illustrated in FIG. 4. The wiring connecting part 71 may cause a short circuit between wires of the plurality of electrodes a (electrode 21a1 and electrode 21a2 in FIG. 4) and between wires of the plurality of electrodes b (electrode 22b1 and electrode 22b2 in FIG. 4) to integrate them. By causing a short circuit, a voltage can be applied to a plurality of electrodes without an error.

As illustrated in FIGS. 3 to 5, an electrode 23 may be disposed on the distal side of the catheter 20 in addition to the electrode part 201. By providing the electrode 23, the intracardiac potential at a place other than the electrode part 201 can be measured.

As illustrated in FIGS. 3 to 5, the electrode 23 may be connected to the electrocardiograph 40. The electrode 23 and the electrocardiograph 40 may be connected via a switch or a resistor, or may be connected without a switch or a resistor as illustrated in FIGS. 3 to 5. Since the electrode 23 and the electrocardiograph 40 are connected without a switch and a resistor, the intracardiac potential can be accurately measured.

As the changeover part 5, a relay switch or a semiconductor switch can be used, for example. Examples of an element of the semiconductor switch include an IGBT, a MOSFET, a thyristor, an element using a SiC semiconductor, and an element using a GaN semiconductor.

As illustrated in FIG. 3, the changeover part 5 includes a switch A (5A) and a switch B (5B), the electrode A (21) is connected to the power supply 2 via the switch A (5A), and the electrode B (22) is connected to the power supply 2 via the switch B (5B). That is, in the circuit diagram illustrated in FIG. 3, the electrode A includes the electrode 21, the electrode B includes the electrode 22, and the electrode 21 and the electrode 22 are connected to the electrocardiograph 40. The switch A includes the switch 5A, the switch B includes the switch 5B, the electrode 21 is connected to the power supply 2 via the switch 5A, and the electrode 22 is connected to the power supply 2 via the switch 5B. As described above, the changeover part 5 includes the switch A (5A) connected to the electrode A and the switch B (5B) connected to the electrode B, whereby the electrode A and the electrode B can be electrically separated from each other, so that each electrode can be independently controlled.

As the switch A and the switch B, different types of switches may be used, but it is preferable to use the same type of switches. By using the same type of switches, control of the intracardiac defibrillation catheter system can be simplified.

As illustrated in FIG. 4, in a case where the electrode A includes a plurality of electrodes a, and the electrode B includes a plurality of electrodes b, it is preferable that the changeover part 5 includes a plurality of switches a connected in parallel to each other and a plurality of switches b connected in parallel to each other, each of the plurality of electrodes a is connected to the power supply 2 via a corresponding one of the plurality of switches a, and each of the plurality of electrodes b is connected to the power supply 2 via a corresponding one of the plurality of switches b. That is, the plurality of electrodes a and the plurality of electrodes b may be connected to the power supply 2 via different switches. That is, in the circuit diagram illustrated in FIG. 4, the electrode A is constituted by the electrode 21 including the electrode 21a1 and the electrode 21a2, the electrode B is constituted by the electrode 22 including the electrode 22b1 and the electrode 22b2, and the electrode 21a1, the electrode 21a2, the electrode 22b1, and the electrode 21b2 are connected to the electrocardiograph 40. The switch A is constituted by a switch 5A including a switch 5A1 and a switch 5A2, the switch B is constituted by a switch 5B including a switch 5B1 and a switch 5B2, the electrode 21a1 is connected to the power supply 2 via the switch 5A1, the electrode 21a2 is connected to the power supply 2 via the switch 5A2, the electrode 21b1 is connected to the power supply 2 via the switch 5B1, and the electrode 21b2 is connected to the power supply 2 via the switch 5B2. As a result, a plurality of electrodes can be electrically separated, so that the intracardiac potential can be measured independently at each electrode. As the plurality of switches a, a plurality of types of switches may be used in combination, but it is preferable to use the same type of switches.

As the plurality of switches b, a plurality of types of switches may be used in combination, but it is preferable to use the same type of switches. By using the same type of switches, control of the intracardiac defibrillation catheter system can be simplified.

In a case where the same type of switches are used as the plurality of switches a and the same type of switches are used as the plurality of switches b, the type of the plurality of switches a and the type of the plurality of switches b may be the same. Thus, control of the intracardiac defibrillation catheter system can be simplified.

The switch a and the switch b may be of a single-pole single-throw type or a multi-pole single-throw type. If each switch is of the single-pole single-throw type, each switch can be operated individually, which makes it easy to apply a voltage to only a specific electrode. If each switch is of a multi-pole single-throw type, multiple switches can be activated in conjunction with one another by one operation, so that the accuracy of a timing at which a voltage is applied to each electrode can be enhanced.

In a case where the electrode A includes a plurality of electrodes a and the electrode B includes a plurality of electrodes b, it is preferable that an electrode selection switch that selects an electrode to which a voltage is applied is connected to the power supply 2. With this configuration, electrical stimulation can be applied only to a specific electrode.

The electrode selection switch may be provided separately from the switches (for example, the switch A and the switch B) constituting the changeover part 5, or at least one of the switches constituting the changeover part 5 may be an electrode selection switch.

The power supply 2 may be provided with a protection circuit that absorbs a high voltage generated when the switch is cut off. This can prevent damage to each switch.

The electrode part 201 and the electrocardiograph 40 may be connected without a switch, but it is preferable to dispose a switch 9 in a connection path between the electrode part 201 and the electrocardiograph 40 as illustrated in FIG. 5. The arrangement of the switch 9 can prevent the electrocardiograph 40 from being damaged by the application of overvoltage. In FIG. 5, the electrode 21 serving as the electrode A is connected to the electrocardiograph 40 via a switch 9a, the electrode 22 serving as the electrode B is connected to the electrocardiograph 40 via a switch 9b, and the switch 9 is constituted by the switch 9a and the switch 9b.

As the switch 9, a relay switch or a semiconductor switch can be used, for example. Examples of an element of the semiconductor switch include an IGBT, a MOSFET, a thyristor, an element using a SiC semiconductor, and an element using a GaN semiconductor.

As illustrated in FIGS. 3 and 4, a resistor may be disposed in a connection path between the electrode part 201 and the electrocardiograph 40. In FIG. 3, the electrode 21 serving as the electrode A is connected to the electrocardiograph 40 via a resistor 81a1, the electrode 22 serving as the electrode B is connected to the electrocardiograph 40 via a resistor 81b1, and the resistor 81 is constituted by the resistor 81a1 and the resistor 81b1. In FIG. 4, the electrode 21a1 constituting the electrode A is connected to the electrocardiograph 40 via a resistor 81a1, the electrode 21a2 constituting the electrode A is connected to the electrocardiograph 40 via a resistor 81a2, the electrode 22b1 constituting the electrode B is connected to the electrocardiograph 40 via a resistor 81b1, the electrode 22b2 constituting the electrode B is connected to the electrocardiograph 40 via a resistor 81b2, and the resistor 81 includes the resistor 81a1, the resistor 81a2, the resistor 81b1, and the resistor 81b2. The arrangement of the resistor 81 can prevent the electrocardiograph 40 from being damaged by the application of overvoltage. Each of the resistors 81 may be 200Ω or less. By setting each of the resistors 81 to 200Ω or less, it is possible to prevent the electrocardiograph 40 from being damaged by the application of overvoltage and to transmit the waveform of the intracardiac potential acquired by the electrode part 201 to the electrocardiograph 40 without causing waveform rounding. The resistor 81 may be 150Ω or less or 100Ω or less, and may be 50Ω or more or 70Ω or more.

The electrocardiograph 40 may be connected to a power supply different from the power supply 2, or may be connected to the power supply 2. By connecting the electrocardiograph 40 to the power supply 2, the configuration of the intracardiac defibrillation catheter system can be simplified.

In a case where the electrocardiograph 40 is connected to the power supply 2, an overvoltage protection circuit protecting the electrocardiograph 40 from overvoltage may be provided in a connection path between the power supply 2 and the electrocardiograph 40. The arrangement of the overvoltage protection circuit can prevent the electrocardiograph 40 from being damaged by the application of overvoltage. The overvoltage protection circuit refers to a circuit having a protecting function of suppressing an overvoltage when an input or an output is in an overvoltage state due to a surge voltage from the outside, an abnormality of a device, or the like.

In a case where the electrocardiograph 40 is connected to the power supply 2, the resistor 81 may be provided in the connection path between the power supply 2 and the electrocardiograph 40 as illustrated in FIGS. 3 and 4. The arrangement of the resistor 81 can prevent the electrocardiograph 40 from being damaged by the application of overvoltage. Each of the resistors 81 may be 200Ω or less. By setting each of the resistors 81 to 200Ω or less, it is possible to prevent the electrocardiograph 40 from being damaged by the application of overvoltage and to transmit the waveform of the intracardiac potential acquired by the electrode part 201 to the electrocardiograph 40 without causing waveform rounding. The resistor may be 150Ω or less, or 100Ω or less. The resistor 81 may be 50Ω or more, or 70Ω or more.

As illustrated in FIGS. 3 to 5, a power supply output control part 61 may be disposed in a connection path between the power supply 2 and the changeover part 5. The power supply output control part 61 outputs the voltage input from the power supply as pulse power. By switching the polarity by the power supply output control part 61, the polarities of the outputs of the electrode A and the electrode B can be inverted.

The power supply output control part 61 may be provided with a switch, and the switch provided in the power supply output control part 61 may have a response speed higher than that of the switch provided in the changeover part 5. As the switch provided in the power supply output control part 61, a semiconductor switch is used, for example.

The power supply 2 may be provided with a switch for safety (safety switch). Due to this configuration, a fail-safe function capable of preventing unintended application of voltage to the patient when, for example, the changeover part 5 fails can be provided to the power supply 2.

The safety switch may be disposed in a connection path between the power supply 2 and the changeover part 5. The number of safety switches is not particularly limited, but it is preferable that at least one safety switch is provided for the plurality of electrodes a and at least one safety switch is provided for the plurality of electrodes b.

As the safety switch, a relay switch or a semiconductor switch can be used, for example. Examples of an element of the semiconductor switch include an IGBT, a MOSFET, a thyristor, an element using a SiC semiconductor, and an element using a GaN semiconductor.

The safety switch may be of a single-pole single-throw type or a multi-pole single-throw type as with the switches A and B described above.

The catheter 20 may be provided with a distal tip 25 at the distal end. The distal tip 25 may have a tapered portion having an outer diameter which decreases toward the distal end of the distal tip 25. As a result, it is possible to improve the insertability of the intracardiac defibrillation catheter system into the body cavity.

Examples of the material constituting the distal tip 25 include a conductive material and a polymer material. In particular, the distal tip 25 can function as an electrode by being made of a conductive material. The hardness of the distal tip 25 may be lower than the hardness of the catheter 20. Thus, when the distal tip 25 comes into contact with the body cavity, the body tissue can be protected.

A handle 26 gripped by a user during operation of the catheter 20 may be provided on the proximal side of the catheter 20. The shape of the handle 26 is not particularly limited, but it is preferable that the handle 26 is formed in a pyramidal shape whose outer diameter decreases toward the distal end of the handle 26 in order to relieve stress concentration at the connection portion between the resin tube and the handle 26. The size of the handle 26 is not particularly limited as long as it is suitable for the user to hold with one hand. The length of the handle 26 is not particularly limited, or may be, for example, 5 cm or more and 20 cm or less. The outermost diameter (circle equivalent diameter) of the handle 26 is not particularly limited, or may be, for example, 1 cm or more and 5 cm or less. Examples of materials usable for the handle 26 include synthetic resin such as ABS or polycarbonate, and foamed plastic such as polyurethane foam.

This application claims the benefit of the priority date of Japanese patent application No. 2022-182528 filed on Nov. 15, 2022. All of the contents of the Japanese patent application No. 2022-182528 are incorporated by reference herein.

### REFERENCE SIGNS LIST

1 Intracardiac defibrillation catheter system
2 Power supply
5 Changeover part
5A, 5A1, 5A2 Switch
5B, 5B1, 5B2 Switch
9, 9a, 9b Switch
20 Catheter
21 Electrode A
22 Electrode B
23 Electrode
25 Distal tip
26 Handle
40 Electrocardiograph
41 Electrocardiogram detection unit
50 Electrocardiographic waveform
51P Peak position of the P wave
51Q Peak position of the Q wave
51R Peak position of the R wave
51S Peak position of the S wave
51 Input part
52 Control part
61 Power supply output control part
71 Wiring connecting part
81, 81a1, 81a2, 81b1, 81b2 Resistor
201 Electrode part

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

1. A control method for an intracardiac defibrillation catheter system comprising:
a catheter extending from a proximal side to a distal side in a longitudinal direction;
an electrode part disposed on a distal side of the catheter;
a power supply for applying a voltage to the electrode part;
an input part for inputting an electrocardiogram; and
a changeover part for switching between a first mode for electrically disconnecting the power supply and the electrode part, and a second mode for electrically connecting the power supply and the electrode part, the changeover part being connected to the power supply, and the electrode part being connected to the power supply via the changeover part,
the control method comprising:
switching the first mode to the second mode with the changeover part when a cardiac potential reaches a peak of a P wave and/or a peak of a Q wave obtained from an electrocardiographic waveform of the electrocardiogram; and
applying a voltage to the electrode part with the power supply when the cardiac potential reaches a peak of an R wave obtained from the electrocardiographic waveform of the electrocardiogram.

2. The control method according to claim 1, wherein the intracardiac defibrillation catheter system further comprises an electrocardiograph connected to the input part.

3. The control method according to claim 2, wherein the electrocardiograph comprises an electrocardiogram detection unit detecting a peak of a P wave and/or a peak of a Q wave and a peak of an R wave from the electrocardiographic waveform of the electrocardiogram obtained from the cardiac potential.

4. The control method according to claim 2, wherein the electrocardiograph is connected to the electrode part.

5. The control method according to claim 1, wherein the electrode part includes an electrode A and an electrode B located proximal to the electrode A.

6. The control method according to claim 5, wherein
the changeover part includes a switch A and a switch B,
the electrode A is connected to the power supply via the switch A, and
the electrode B is connected to the power supply via the switch B.

7. The control method according to claim 5, wherein
the electrode A includes a plurality of electrodes a,
the electrode B includes a plurality of electrodes b,
the changeover part includes a plurality of switches a connected in parallel to each other and a plurality of switches b connected in parallel to each other,
each of the plurality of electrodes a is connected to the power supply via a corresponding one of the plurality of switches a, and
each of the plurality of electrodes b is connected to the power supply via a corresponding one of the plurality of switches b.

8. The control method according to claim 7, wherein the intracardiac defibrillation catheter system further comprises an electrode selection switch connected to the power supply for selecting an electrode to which the voltage is applied.

9. The control method according to claim 2, wherein the intracardiac defibrillation catheter system further comprises a switch disposed in a connection path between the electrode part and the electrocardiograph.

10. The control method according to claim 2, wherein the intracardiac defibrillation catheter system further comprises a resistor of 200Ω or less disposed in a connection path between the electrode part and the electrocardiograph.

11. The control method according to claim 2, wherein
the electrocardiograph is connected to the power supply, and
the intracardiac defibrillation catheter system further comprises an overvoltage protection circuit provided in a connection path between the power supply and the electrocardiograph for protecting the electrocardiograph from overvoltage.

12. The control method according to claim 1, wherein the intracardiac defibrillation catheter system further comprises a power supply output control part disposed in a connection path between the power supply and the changeover part.
